(19) United States
(12) Patent Application Publication
(10) Pub. No.: US 2021/0109089 A1
(43) Pub. Date: Apr. 15, 2021

(54) METHOD FOR CONSTRUCTING CHROMATOGRAPHY TEST STRIP FOR TRIAZOPHOS BASED ON MOLECULAR IMPRINTING AND ELECTROSPINNING

(71) Applicant: Institute of Quality Standard and Testing Technology for Agro-products, Chinese Academy of Agricultu, Beijing (CN)

(72) Inventors: Yongxin She, Beijing (CN); Sihui Hong, Beijing (CN); Miao Wang, Beijing (CN); Zhen Cao, Beijing (CN); Yahui He, Beijing (CN); Jing Wang, Beijing (CN)

(21) Appl. No.: 16/911,824

(22) Filed: Jun. 25, 2020

(30) Foreign Application Priority Data
Oct. 10, 2019 (CN) 201910959017.2

(51) Int. Cl. G01N 33/531; D01D 1/02; D01D 5/00; D01F 8/02; D01F 8/04

(52) U.S. Cl. CPC G01N 33/531; D01D 1/02; D01D 5/003; D01F 8/04; D01D 5/0092; D01D 5/0076; D01F 8/02; D01D 5/0069

(57) ABSTRACT

The present invention relates to a method for constructing a chromatography test strip for triazophos based on molecular imprinting and electrospinning. The present invention combines electrospinning, molecular imprinting and the immunochromatography test strip technology. Molecularly imprinted T-line (detection limit) is prepared on an NC membrane by electrospinning, and goat anti-mouse IgG is used as C-line (quality control line). With fluorescence changes occurring when triazophos hapten-murine IgG/fluorescein isothiocyanate conjugate (THBu-IgG-FITC) fluorescent probe directly competes with the target triazophos to bind to the molecularly imprinted binding site, a chromatography-fluorescence detection method for triazophos based on molecular imprinting and electrospinning is established. The functional material adsorbing triazophos provided by the present invention adopts a virtual template to avoid template leakage, and can be used in immunochromatography to replace a biological antibody. The functional material has higher selectivity, higher stability, longer service life, and stronger resistance to adverse environment.

### CROSS-REFERENCE TO RELATED APPLICATIONS

The present application claims priority to Chinese Application No. 201910959017.2 filed on Oct. 10, 2019, the disclosure of which is incorporated herein by reference.

### TECHNICAL FIELD

The present invention relates to the technical field of food-safety detection, and in particular, to a method for constructing a chromatography test strip for triazophos based on molecular imprinting and electrospinning.

### BACKGROUND

Triazophos, a toxic, broad-spectrum organophosphorus insecticide, is widely applied to grains, fruits and vegetables. Triazophos tends to remain in the environment due to its outstanding chemical stability and long half-life, causing potential hazard to the environment and human health. China has banned the use of triazophos on vegetables since Dec. 31, 2016. At present, the detection technology for triazophos mainly includes the confirmation technology and the immunoassay technology, but these technologies usually have many disadvantages, such as expensive equipment, long analysis time, and complicated antibody preparation. Therefore, it is of great practical significance to design and synthesize a biomimetic recognition material with strong specificity and excellent stability at low cost, and to establish a sensitive, simple, fast, stable, and inexpensive detection method.

A detection technology based on immunochromatography test strip is a solid-phase labeling immunoassay technology that combines the monoclonal antibody technology, immunolabeling, immunochromatography and the like, and is applied to the qualitative, semi-quantitative and quantitative analysis of an antigen, antibody and hapten. This method has become one of the most common immunoassay methods, as it is convenient, fast, highly-specific, low-cost and simple, and requires no professionals and large and expensive equipment. However, a regular immunochromatography method requires the use of an antibody, resulting in disadvantages such as high cost, high storage conditions, and a need for sacrificing animals. It is expected to overcome these disadvantages by replacing an antibody with a molecularly imprinted polymer. As of now, the only biomimetic immunochromatography technology based on molecular imprinting is the competitive colloidal gold test strip for atrazine constructed by Xie Rong. In this method, the larger size of molecularly imprinted microspheres may cause poor chromatography and thus affect the sensitivity of the experiment. In addition, as there is no antibody corresponding to molecular imprinting, this method adopts two test strips for detection and quality control respectively, resulting in errors and results of lower accuracy.

In order to overcome the above disadvantages, the present invention intends to adopt a new biomimetic immunochromatography mode based on molecular imprinting to directly attach a molecularly imprinted polymer to an NC membrane (nitrocellulose membrane). However, if the molecularly imprinted polymer is directly fixed to the NC membrane by scribing, on the one hand, the molecularly imprinted polymer is easy to be migrated from the membrane during the chromatography process, and on the other hand, the specific binding site on the molecularly imprinted polymer does not tend to be exposed on its surfaces, resulting in a decrease in experimental sensitivity. In order to solve this problem, the inventors intend to prepare a molecularly imprinted nanofiber membrane on an NC membrane by electrospinning. The molecularly imprinted polymer can be tightly fixed, and can also fully expose its binding site to increase the contact area with a target, thereby improving the mass transfer rate.

Electrospinning is a unique fiber manufacturing process that produce fine fibers from a polymer solution or melt with an electrostatic force. The fibers produced by this process have a smaller diameter (from micrometers to nanometers) and a larger surface area than that produced by a traditional spinning process. During the electrospinning process, the polymer solution held at the end of the capillary by a surface tension is subjected to an electric field, and the electric field induces a charge on the liquid surface. When the applied electric field reaches a critical value, the electrostatic repulsion counteracts the surface tension, and a charged jet of the solution is ejected from the tip of Taylor cone. An unstable eruption occurs in the space between the capillary tip and the collector, during which the solvent evaporates and fibers are formed on the collector. The electrospinning fiber, which has a larger specific surface area, a higher porosity, and stronger physical and mechanical properties than a conventional fiber, has been widely used in fields of tissue engineering scaffolds, drug delivery, filtration, healthcare, biotechnology, environmental engineering, defense, and security.

The combination of electrospinning and molecular imprinting enables the specific adsorption of the electrospinning fiber membrane, and also improves the specific surface area of the molecularly imprinted polymer, the adsorption capacity, and the mass transfer rate. In recent years, the preparation of a molecularly imprinted membrane by electrospinning has attracted widespread attention. At present, the method for preparing a molecularly imprinted fiber membrane by electrospinning mainly includes embedding and direct electrospinning. The present invention adopts the embedding method, that is, the molecularly imprinted microspheres are prepared by the precipitation method, and then directly mixed with an electrospinning solution to prepare molecularly imprinted nanofibers. So far, there has been no report about the combination of electrospinning with immunochromatography.

The invention combines electrospinning, molecular imprinting and a detection technology based on immunochromatography test strip. Molecularly imprinted T-line (detection limit) is prepared on an NC membrane by electrospinning, and goat anti-mouse IgG is used as C-line (quality control line). With fluorescence changes occurred when triazophos hapten-murine IgG/fluorescein isothiocyanate conjugate (THBu-IgG-FITC) fluorescent probe directly competes with the target triazophos to bind to the molecularly imprinted binding site, a chromatography-fluorescence detection method based on molecular imprinting and electrospinning for triazophos is established to detect the triazophos residue.

### BRIEF SUMMARY

In order to overcome disadvantages of traditional detection methods for triazophos, such as long detection time, expensive equipment, and complicated preparation for specific antibodies, the present invention provides a method for constructing a chromatography test strip for triazophos based on molecular imprinting and electrospinning, and a chromatography assay method for triazophos based on a electrospinning membrane fabrication technology.

To achieve the above objective, the following technical solutions are adopted.

A chromatography assay method for triazophos based on a electrospinning membrane fabrication technology includes the following steps:

1. Synthesis of Hapten

(1) Synthesis of O-ethyl dichlorothiophosphate (TZM-1): 68 g (about 0.4 mol) of thiophosphoryl chloride (PSCl3) is weighed and added to a three-necked flask with a low-temperature thermometer, and the liquid is cooled to −10° C. to −5° C. in an ice-brine bath. 55 g (about 1.2 mol) of absolute ethyl alcohol is added dropwise with vigorous stirring at a rate that is strictly controlled so that the temperature of the reaction solution is always not higher than 0° C. After the dropwise addition is completed, the reaction is continued at 10° C. for 2 h. After the reaction is completed, the reaction solution is washed with (0±5)° C. distilled water (100 ml×2). The oil phase is separated, dried over anhydrous Na2SO4, and then distilled under reduced pressure with a water aspirator. Fraction at 65° C. to 75° C. is collected to obtain a colorless, transparent and oily liquid (51.8 g; yield 72.3%, calculated based on thiophosphoryl chloride).
(2) Synthesis of O-ethyl-0-[3-(1-phenyl-1, 2, 4-triazolyl)chlorothiophosphate (TZM-2): 36 g (about 0.2 mol) of TZM-1 is weighed and added to a 250 ml three-necked flask. About 16 g (about 0.1 mol) of 1-phenyl-1, 2, 4-triadimenol is added with stirring, and then about 15 ml of TEA and 80 ml of DCM are added. After all solids are dissolved, the resulting solution is cooled to a temperature lower than 20° C. in an ice water bath. Then a trace amount of catalyst is added, and 55 ml of a 2 mol/L NaOH aqueous solution is added dropwise. The reaction continues for 1 h. After the reaction is completed, 50 ml of 5% NaOH iced aqueous solution is added. The resulting solution is shaken, and the water phase is removed. The oil phase is washed with ice water to neutrality, dried over anhydrous Na2SO4, and concentrated under reduced pressure to obtain a small amount of brown oily substance. Petroleum ether (50 ml×2) is added to the oily substance for extraction, and the extract is concentrated under reduced pressure to obtain a yellow liquid (10.6 g; yield 35%, calculated based on triadimenol).
(3) Synthesis of triazophos hapten: 1.03 g (about 10 mmol) of 4-aminobutyric acid is weighed and dissolved in 10 ml of a NaOH solution (1 mol/L), and the resulting solution is cooled to 0° C. to 10° C. in an ice water bath. 1.51 g (about 5 mmol) of TZM-2 dissolved in 10 ml of dioxane is slowly added with stirring, a trace amount of catalyst is added, and 10 ml of a NaOH aqueous solution (1 mol/L) is added dropwise. The solution is warmed to 15° C. to 25° C. for 4 h of reaction. After the reaction is completed, 50 ml of water is added, and the reaction solution is washed with petroleum ether (40 ml×2), and the petroleum ether phase is removed. pH of the water phase is adjusted to about 3 with 2 mol/L HCL, and ethyl acetate (40 ml×2) is added for extraction. The extract is washed with a small amount of water, dried over anhydrous Na2SO4, and concentrated under reduced pressure. The residue is sealed and stored overnight at 4° C., and a colorless product is precipitated. The precipitate is recrystallized with an ethyl acetate-petroleum ether system, filtered out, and dried to obtain 0.52 g of a white solid (THBu, yield 27%, calculated based on intermediate TZM-2).

2. Preparation of THBu-IgG-FITC fluorescent probe

(1) 9.43 mg of triazophos hapten (0.025 mmol) is weighed and dissolved in 0.5 ml of DMF.
(2) 8.63 mg of NHS (0.075 mmol) is weighed and added to the solution prepared in step 1, and the resulting mixture is stirred at room temperature for 15 min.
(3) 7.73 mg of DCC (0.0375 mmol) is weighed and dissolved in 0.5 ml of DMF, and the obtained solution is added to the solution prepared in step 2 dropwise. The resulting mixture is stirred overnight at room temperature and then centrifuged at 4,000 rpm/min for 10 min.
(4) 200 μL of the supernatant in step 3 is pipetted and slowly added to 1 ml of CBS solution (0.01 mol/L) in which 10 mg of mouse IgG is dissolved, and the resulting solution is stirred at 20° C. for 4 h.
(5) 2.95 mg of FITC is weighed and dissolved in 2.95 ml of CBS (0.05 mol/L, pH=9.6), the obtained solution is added to the reaction solution in step 4 dropwise in the dark. Then the reaction solution is slowly stirred at 4° C. for 8 h in the dark.
(6) The synthesized THBu-IgG-FITC fluorescent probe is dialyzed in a 0.01 mol/L PBS (pH=7.4) solution at 4° C. until the dialysate is clear, and stored at 4° C. The fluorescent probe is not suitable for long-term storage, and should be used as soon as possible.

3. Preparation of Molecularly Imprinted Microspheres

29.4 mg (0.1 mmol) of triazolone (template) is weighed and added to a 100 ml round-bottom flask, and 20 ml of acetonitrile (pore-forming agent) is added to dissolve the template. Then 51 μL (0.6 mmol) of MAA (functional monomer) is added, and the mixture is shaken at room temperature for 30 min of prepolymerization. 319.3 μL (1.0 mmol) of TRIM (crosslinking agent) and 30 mg of AIBN (initiator) are then added, and the tube is sealed immediately after 2 min of nitrogen charge. The polymerization reaction is conducted in a 60° C. water bath for 24 h. After the polymerization is completed, the reaction solution is taken out and centrifuged, and the supernatant is removed. Then the resulting precipitate is dispersed in methanol and then centrifuged to remove the unreacted reactant. The obtained polymer is wrapped with a filter paper, and placed in a Soxhlet extractor for extracting the template with a methanol:acetic acid (9:1, v/v) solution.

4. Construction of Chromatography Test Strip

A sample pad is treated with a sample pad treatment solution (0.5% Tween-0.02 M pH 7.2 PB buffer), then dried, and cut into strips. A secondary antibody (goat anti-mouse IgG) is drawn on an NC membrane at a flow rate of 1 μL/cm by a scriber and dried at 37° C. Then a test strip is assembled as follows: as T-line needs to be spun on an aluminum foil (NC membrane is non-conductive), the NC membrane is cut along a line 5 mm below C-line, and an aluminum foil of 1 mm width is placed between the obtained two NC membranes; the upper and lower NC membranes and the middle aluminum foil are pasted on a black fluorescent board, with T-line and C-line being 5 mm apart from each other; and then an absorbent pad and the sample pad are pasted on the upper and lower sides of the NC membrane respectively, with each pad overlapping with the NC membrane by 1 mm. The assembled test strip is shown in FIG. 1 (a).

5. Preparation of molecularly imprinted T-line on an NC membrane by electrospinning

(1) Preparation of an electrospinning solution Preparation of a CA electrospinning matrix solution: A certain amount of CA powder is weighed and added to acetone for preparing a 120 mg/ml CA-acetone solution, and the obtained solution is shaken at 50° C. in a water bath for 5 h until CA is completely dissolved. Preparation of an MIP dispersion solution for triazolone: A certain amount of MIPs is weighed and added to acetone for preparing a 20 mg/ml MIP dispersion solution, and the obtained solution is subjected to ultrasonic dispersion at room temperature for 50 min until MIPs are completely and evenly dispersed in acetone. Mixing of the CA electrospinning matrix solution with the MIP dispersion solution: 111 μL of 20 mg/ml MIP dispersion solution is added to 1 ml of 120 mg/ml CA electrospinning matrix solution, then 7 μL of 10% Tween solution is added, and the resulting solution is shaken in a 50° C. water bath for 120 min and subjected to ultrasonic dispersion for 30 min at room temperature to obtain an uniform MIP electrospinning solution for triazolone.
(2) Preparation of molecularly imprinted T-line on an NC membrane by electrospinning:

An electrospinning device made in laboratory, with an automatic microflow pump, a 5 ml syringe, a height-adjusting frame, a jet needle (22 G), a receiving plate, and a high-voltage power supply, is adopted. Before spinning, the grounding is checked, and the temperature and humidity are recorded. The prepared MIP electrospinning solution is drawn into the syringe, the distance between the jet needle and the receiving plate is adjusted to 13 cm, and the flow rate of the microflow pump is set as 12 μL/min, and the high voltage as 12.0 kV. After the fiber extrusion is stable, the assembled test strip is placed on the receiving plate (ensuring that it is placed at the same position each time), and one end of the T-line aluminum foil is clamped with a negative electrode. 20 min later, molecularly imprinted nanofibers evenly cover T-line without covering other parts of the test strip that are not conductive. The obtained test strip is dried in an oven at 37° C., then cut into smaller strips having a width of 3.5 mm by a slitter, and stored in a desiccator at room temperature. The molecularly imprinted test strips are obtained.

6. Experimental principle: A molecularly imprinted polymer, instead of an artificial antibody, is fixed on an NC membrane as T-line by electrospinning, and a secondary antibody is fixed as C-line by a scriber. As shown in FIG. 1 (b), when the target and the THBu-IgG-FITC fluorescent probe are added dropwise to the sample pad, the solution moves on the NC membrane by capillary action. Both the target triazophos and the triazophos hapten on the THBu-IgG-FITC probe can bind to the molecularly imprinted polymer on T-line, and IgG on the probe can bind to the secondary antibody on C-line. When moving to T-line, the target and the fluorescent probe compete to bind to the specific binding site on the molecularly imprinted polymer, causing the fluorescence intensity on T-line to be inversely proportional to the concentration of the target, and as the remaining target and probe continue to move to C-line, the IgG on the probe binds to the secondary antibody to achieve the quality control. A fluorescence immunoassay analyzer (wavelength for excitation: 450 nm to 470 nm, wavelength for receiving: 525 nm) is used to read the fluorescence values of C-line and T-line, and a qualitative and quantitative assay is performed according to the fluorescence intensity at T-line and the T/C value.

7. Experimental process

(1) Preparation of test strips: Molecularly imprinted test strips are assembled according to step 4 and 5, and blocked with a blocking buffer (0.25% PVP+0.25% BSA+5% sucrose), dried at 37° C., and stored in a desiccator at room temperature.
(2) Competitive reaction: 100 μL of 10-fold-diluted THBu-IgG-FITC fluorescent probe (diluted with 0.01 M PBS) is added dropwise to the sample well of the test strip for chromatography, and 3 min later, the test strip is dried in a 37° C. oven for 15 min. Then 100 μL of triazophos standard solution or sample is added for chromatography, and 3 min later, the fluorescence detection is performed.
(3) Detection: The T/C value is read with a single-channel fluorescence immunoassay analyzer, and the content of triazophos is calculated according to a standard curve.

The advantages and beneficial effects of the present invention are as follows:

1. The functional material adsorbing triazophos provided by the present invention adopts a virtual template to avoid template leakage, and can be used in immunochromatography to replace a biological antibody. The functional material, prepared by a chemical process, has higher selectivity, higher stability, longer service life, and stronger resistance to adverse environment. Therefore, the present invention overcomes the disadvantages of a conventional biological antibody, such as long preparation cycle, high proneness to deactivation, and high cost.

2. In the present invention, a composite nanomembrane of nanofibers and molecularly imprinted microspheres is synthesized by a electrospinning membrane fabrication technology, a triazophos hapten-IgG-FITC fluorescent probe is prepared, a nanomembrane chromatography test strip specifically recognizing triazophos is preliminarily developed by combining electrospinning, molecular imprinting and a detection technology based on immunochromatography test strip, and a new method for rapidly detecting triazophos is established. The prepared test strip is linearly correlated with the concentration of triazophos in a range of 20 μg/L to 500 μg/L (y=−0.2638x+0.8695, R2=0.954), with a detection limit of 20 μg/L and a detection time only of 30 min. The method is fast, simple, portable, and suitable for on-site rapid detection. It is expected to achieve the qualitative and quantitative analysis of triazophos residue in an actual sample with this method in the future. Moreover, in the present invention, electrospinning is used for the first time to prepare a molecularly imprinted immunochromatography nanomembrane, which improves the stability and recognition performance of T-line and presents a new clue for the immunochromatography test paper technology based on a novel biomimetic recognition material.

### BRIEF DESCRIPTION OF THE DRAWINGS

FIG. 1 is a structure diagram of molecularly imprinted electrospinning test strip (a) and a flow chart of directly-competitive fluorescence detection (b); and

FIG. 2 is a standard curve of a test strip for triazophos.

### DETAILED DESCRIPTION

To enable a person skilled in the art to better understand the present invention, the technical solutions of the present invention is further described below with reference to the accompanying drawings and examples.

1. Synthesis of Hapten

(1) Synthesis of O-ethyl dichlorothiophosphate (TZM-1): 68 g (about 0.4 mol) of thiophosphoryl chloride (PSCl3) was weighed and added to a three-necked flask with a low-temperature thermometer, and the liquid was cooled to −10° C. to −5° C. in an ice-brine bath. 55 g (about 1.2 mol) of absolute ethyl alcohol was added dropwise with vigorous stirring at a rate that was strictly controlled so that the temperature of the reaction solution was always not higher than 0° C. After the dropwise addition was completed, the reaction was continued at 10° C. for 2 h. After the reaction was completed, the reaction solution was washed with (0±5)° C. distilled water (100 ml×2). The oil phase was separated, dried over anhydrous Na2SO4, and then distilled under reduced pressure with a water aspirator. Fraction at 65° C. to 75° C. was collected to obtain a colorless, transparent and oily liquid (51.8 g; yield 72.3%, calculated based on thiophosphoryl chloride).
(2) Synthesis of O-ethyl-O-[3-(1-phenyl-1, 2, 4-triazolyl)chlorothiophosphate (TZM-2): 36 g (about 0.2 mol) of TZM-1 was weighed and added to a 250 ml three-necked flask. About 16 g (about 0.1 mol) of 1-phenyl-1, 2, 4-triadimenol was added with stirring, and then about 15 ml of TEA and 80 ml of DCM were added. After all solids were dissolved, the resulting solution was cooled to a temperature lower than 20° C. in an ice water bath. Then a trace amount of catalyst was added, and 55 ml of a 2 mol/L NaOH aqueous solution was added dropwise. The reaction continued for 1 h. After the reaction was completed, 50 ml of 5% NaOH iced aqueous solution was added. The resulting solution was shaken, and the water phase was removed. The oil phase was washed with ice water to neutrality, dried over anhydrous Na2SO4, and concentrated under reduced pressure to obtain a small amount of brown oily substance. Petroleum ether (50 ml×2) was added to the oily substance for extraction, and the extract was concentrated under reduced pressure to obtain a yellow liquid (10.6 g; yield 35%, calculated based on triadimenol).
(3) Synthesis of triazophos hapten: 1.03 g (about 10 mmol) of 4-aminobutyric acid was weighed and dissolved in 10 ml of a NaOH solution (1 mol/L), and the resulting solution was cooled to 0° C. to 10° C. in an ice water bath. 1.51 g (about 5 mmol) of TZM-2 dissolved in 10 ml of dioxane was slowly added with stirring, a trace amount of catalyst was added, and 10 ml of a NaOH aqueous solution (1 mol/L) was added dropwise. The solution was warmed to 15° C. to 25° C. for 4 h of reaction. After the reaction was completed, 50 ml of water was added, and the reaction solution was washed with petroleum ether (40 ml×2), and the petroleum ether phase was removed. pH of the water phase was adjusted to about 3 with 2 mol/L HCL, and ethyl acetate (40 ml×2) was added for extraction. The extract was washed with a small amount of water, dried over anhydrous Na2SO4, and concentrated under reduced pressure. The residue was sealed and stored overnight at 4° C., and a colorless product was precipitated. The precipitate was recrystallized with an ethyl acetate-petroleum ether system, filtered out, and dried to obtain 0.52 g of a white solid (THBu, yield 27%, calculated based on intermediate TZM-2).

2. Preparation of THBu-IgG-FITC fluorescent probe

(1) 9.43 mg of triazophos hapten (0.025 mmol) was weighed and dissolved in 0.5 ml of DMF.
(2) 8.63 mg of NHS (0.075 mmol) was weighed and added to the solution prepared in step 1, and the resulting mixture was stirred at room temperature for 15 min.
(3) 7.73 mg of DCC (0.0375 mmol) was weighed and dissolved in 0.5 ml of DMF, and the obtained solution was added to the solution prepared in step 2 dropwise. The resulting mixture was stirred overnight at room temperature and then centrifuged at 4,000 rpm/min for 10 min.
(4) 200 μL of the supernatant in step 3 was pipetted and slowly added to 1 ml of CBS solution (0.01 mol/L) in which 10 mg of mouse IgG was dissolved, and the resulting solution was stirred at 20° C. for 4 h.
(5) 2.95 mg of FITC was weighed and dissolved in 2.95 ml of CBS (0.05 mol/L, pH=9.6), the obtained solution was added to the reaction solution in step 4 dropwise in the dark. Then the reaction solution was slowly stirred at 4° C. for 8 h in the dark.
(6) The synthesized THBu-IgG-FITC fluorescent probe was dialyzed in a 0.01 mol/L PBS (pH=7.4) solution at 4° C. until the dialysate was clear, and stored at 4° C. The fluorescent probe is not suitable for long-term storage, and should be used as soon as possible.

3. Preparation of molecularly imprinted microspheres: 29.4 mg (0.1 mmol) of triazolone (template) was weighed and added to a 100 ml round-bottom flask, and 20 ml of acetonitrile (pore-forming agent) was added to dissolve the template. Then 51 μL (0.6 mmol) of MAA (functional monomer) was added, and the mixture was shaken at room temperature for 30 min of prepolymerization. 319.3 μL (1.0 mmol) of TRIM (crosslinking agent) and 30 mg of AIBN (initiator) were then added, and the tube was sealed immediately after 2 min of nitrogen charge. The polymerization reaction was conducted in a 60° C. water bath for 24 h. After the polymerization was completed, the reaction solution was taken out and centrifuged, and the supernatant was removed. Then the resulting precipitate was dispersed in methanol and then centrifuged to remove the unreacted reactant. The obtained polymer was wrapped with a filter paper, and placed in a Soxhlet extractor for extracting the template with a methanol:acetic acid (9:1, v/v) solution.

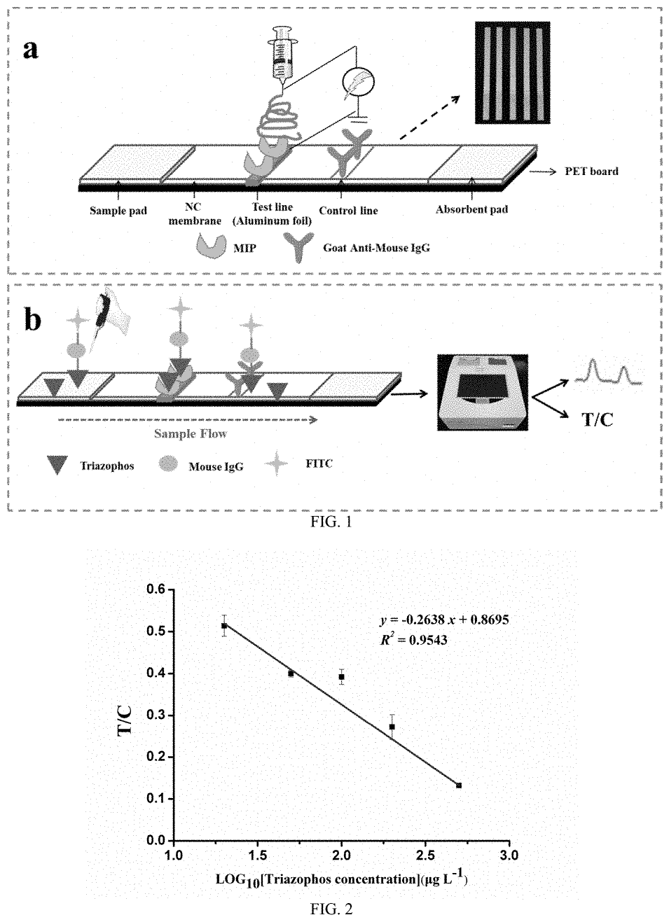

4. Construction of chromatography test strip: A sample pad was treated with a sample pad treatment solution (0.5% Tween-0.02 M pH 7.2 PB buffer), then dried, and cut into strips. A secondary antibody (goat anti-mouse IgG) was drawn on an NC membrane at a flow rate of 1 μL/cm by a scriber and dried at 37° C. Then a test strip was assembled as follows: as T-line needed to be spun on an aluminum foil (NC membrane is non-conductive), the NC membrane was cut along a line 5 mm below C-line, and an aluminum foil of 1 mm width was placed between the obtained two NC membranes; the upper and lower NC membranes and the middle aluminum foil were pasted on a black fluorescent board, with T-line and C-line being 5 mm apart from each other; and then an absorbent pad and the sample pad were pasted on the upper and lower sides of the NC membrane respectively, with each pad overlapping with the NC membrane by 1 mm. The assembled test strip is shown in FIG. 1 (a).

5. Preparation of molecularly imprinted T-line on an NC membrane by electrospinning

(1) Preparation of an electrospinning solution Preparation of a CA electrospinning matrix solution: A certain amount of CA powder was weighed and added to acetone for preparing a 120 mg/ml CA-acetone solution, and the obtained solution was shaken at 50° C. in a water bath for 5 h until CA was completely dissolved. Preparation of an MIP dispersion solution for triazolone: A certain amount of MIPs was weighed and added to acetone for preparing a 20 mg/ml MIP dispersion solution, and the obtained solution was subjected to ultrasonic dispersion at room temperature for 50 min until MIPs were completely and evenly dispersed in acetone. Mixing of the CA electrospinning matrix solution with the MIP dispersion solution: 111 μL of 20 mg/ml MIP dispersion solution was added to 1 ml of 120 mg/ml CA electrospinning matrix solution, then 7 μL of 10% Tween solution was added, and the resulting solution was shaken in a 50° C. water bath for 120 min and subjected to ultrasonic dispersion for 30 min at room temperature to obtain an uniform MIP electrospinning solution for triazolone.
(2) Preparation of molecularly imprinted T-line on an NC membrane by electrospinning: An electrospinning device made in laboratory, with an automatic microflow pump, a 5 ml syringe, a height-adjusting frame, a jet needle (22 G), a receiving plate, and a high-voltage power supply, was adopted. Before spinning, the grounding was checked, and the temperature and humidity were recorded. The prepared MIP electrospinning solution was drawn into the syringe, the distance between the jet needle and the receiving plate was adjusted to 13 cm, and the flow rate of the microflow pump was set as 12 μL/min, and the high voltage as 12.0 kV. After the fiber extrusion was stable, the assembled test strip was placed on the receiving plate (ensuring that it was placed at the same position each time), and one end of the T-line aluminum foil was clamped with a negative electrode. 20 min later, molecularly imprinted nanofibers evenly covered T-line without covering other parts of the test strip that are not conductive. The obtained test strip was dried in an oven at 37° C., then cut into smaller strips having a width of 3.5 mm by a slitter, and stored in a desiccator at room temperature. The molecularly imprinted test strips were obtained.

6. Experimental principle: A molecularly imprinted polymer, instead of an artificial antibody, is fixed on an NC membrane as T-line by electrospinning, and a secondary antibody is fixed as C-line by a scriber. As shown in FIG. 1 (b), when the target and the THBu-IgG-FITC fluorescent probe are added dropwise to the sample pad, the solution moves on the NC membrane by capillary action. Both the target triazophos and the triazophos hapten on the THBu-IgG-FITC probe can bind to the molecularly imprinted polymer on T-line, and IgG on the probe can bind to the secondary antibody on C-line. When moving to T-line, the target and the fluorescent probe compete to bind to the specific binding site on the molecularly imprinted polymer, causing the fluorescence intensity on T-line to be inversely proportional to the concentration of the target, and as the remaining target and probe continue to move to C-line, the IgG on the probe binds to the secondary antibody to achieve the quality control. A fluorescence immunoassay analyzer (wavelength for excitation: 450 nm to 470 nm, wavelength for receiving: 525 nm) is used to read the fluorescence values of C-line and T-line, and a qualitative and quantitative assay is performed according to the fluorescence intensity at T-line and the T/C value.

7. Experimental process

(1) Preparation of test strips: Molecularly imprinted test strips were assembled according to step 4 and 5, and blocked with a blocking buffer (0.25% PVP+0.25% BSA+5% sucrose), dried at 37° C., and stored in a desiccator at room temperature.
(2) Competitive reaction: 100 μL of 10-fold-diluted THBu-IgG-FITC fluorescent probe (diluted with 0.01 M PBS) was added dropwise to the sample well of the test strip for chromatography, and 3 min later, the test strip was dried in a 37° C. oven for 15 min. Then 100 μL of triazophos standard solution or sample was added for chromatography, and 3 min later, the fluorescence detection was performed.
(3) Detection: The T/C value was read with a single-channel fluorescence immunoassay analyzer, and the content of triazophos was calculated according to a standard curve. It can be seen from FIG. 2 that the minimum detection limit of this assay method for triazophos is 20 μg/L, meeting the detection requirement.

The foregoing examples are merely illustrative of preferred implementations of the present invention, and the description thereof is more specific and detailed, but should not be construed as limiting the patent scope of the present invention. It should be noted that several variations, improvements and replacements may be made by those of ordinary skill in the art without departing from the conception of the present invention, but such variations, improvements and replacements should fall within the protection scope of the present invention. Therefore, the patent protection scope of the present invention should be subject to the appended claims.

1. (canceled)

2. A method for constructing a chromatography test strip for triazophos comprising the steps of:
constructing a chromatography test strip, comprising:
treating a sample pad with a sample pad treatment solution;
drying the sample pad;
cutting the sample pad into strips;
drawing a secondary antibody on a nitrocellulose (NC) membrane at a predetermined flow rate by a scriber;
drying the NC membrane at a first temperature;
assembling the test strip, comprising:
cutting the NC membrane along a line a prescribed dimension below a quality control line (C-line);
placing an aluminum foil strip between the cut NC membranes including an upper NC membrane and a lower NC membrane;
pasting the upper NC membrane, the lower NC membrane, and the middle aluminum foil on to a fluorescent board, with a test line (T-line) being separated by a prescribed distance from each other; and
pasting an absorbent pad and the sample pad on upper NC membrane and the lower NC membrane, respectively, with each of the absorbent pad and the sample pad overlapping the NC membrane by a prescribed overlap distance;
preparing, by electrospinning, a molecularly imprinted T-line on the NC membranes, comprising:
preparing an electrospinning solution, comprising:
preparing a cellulose acetate (CA) electrospinning matrix solution, comprising:
adding a weighted measure of a CA powder to acetone for CA-acetone solution;
agitating the CA-acetone solution at a second prescribed temperature in a water bath for a set duration, the CA being dissolved in the CA-acetone solution; and
preparing a molecularly imprinted polymer (MIP) dispersion solution for triazolone, comprising:
adding a weighted measure of MIP to acetone;
subjecting the MIP dispersion solution to ultrasonic dispersion at a third prescribed temperature, the MIP being dissolved and evenly dispersed in the acetone; and
mixing a prescribed volume of the CA electrospinning matrix solution with the MIP dispersion solution and being agitated in a water bath a third prescribed temperature to yield the electrospinning solution;
drawing the electrospinning solution into a syringe of an electrospinning device;
placing the assembled test strip on to a receiving plate of the electrospinning device; and
clamping a first end of the T-line to a negative electrode, molecularly imprinted nanofibers evenly covering the T-line without covering other non-conductive parts of the test strip.

3. The method of claim 2, wherein the sample pad treatment solution is a 0.5% polysorbate surfactant buffer.

4. The method of claim 2, wherein the predetermined flow rate for drawing the secondary antibody on the NC membrane is 1 μL/cm.

5. The method of claim 2, wherein the secondary antibody is a goat anti-mouse IgG.

6. The method of claim 2, wherein the first temperature is 37° C.

7. The method of claim 2, wherein the prescribed dimension below the quality control line is 5 mm.

8. The method of claim 2, wherein the aluminum foil strip is 1 mm in width.

9. The method of claim 2, wherein the prescribed distance between the C-line and the T-line is 5 mm.

10. The method of claim 2, wherein the prescribed overlap distance is 1 mm.

11. The method of claim 2, wherein the CA-acetone solution has a 120 mg/ml CA concentration.

12. The method of claim 2, wherein the second prescribed temperature is 50° C.

13. The method of claim 2, wherein the MIP dispersion solution has a 20 mg/ml MIP concentration.

14. The method of claim 2, wherein mixing the prescribed volume of the CA electrospinning matrix solution with the MIP dispersion solution includes:
adding 111 μL of the MIP dispersion to 1 ml of the CA electrospinning matrix solution; and
adding 7 μL of a 10% polysorbate surfactant buffer.

15. The method of claim 2, wherein the electrospinning solution is subject to ultrasonic dispersion for a predetermined duration at room temperature.

16. The method of claim 2, wherein the electrospinning device includes an automatic microflow pump, the syringe, a height-adjusting frame, the jet needle (22 G), the receiving plate, and a high-voltage power supply. Before spinning, the grounding is checked, and the temperature and humidity are recorded;

17. The method of claim 16, further comprising:
adjusting the distance between the jet needle and a receiving plate of the electrospinning device to 13 cm;
adjusting the flow rate of the microflow pump to 12 μL/min; and
adjusting the high-voltage power supply to 12.0 kV.

18. The method of claim 2, further comprising:
drying the test strips upon the molecularly imprinted nanofibers covering the T-line; and
cutting the test strip into a plurality of smaller strips.

19. The method of claim 18, wherein the smaller strips have a width dimension of 3.5 mm.

20. The method of claim 18, further comprising storing the smaller strips in a dessicator at room temperature.
